(19) United States
(12) Patent Application Publication
(10) Pub. No.: US 2026/0363814 A1
(43) Pub. Date: Nov. 19, 2026

(54) SYSTEM AND METHOD FOR DIGITAL ANATOMICAL RECONSTRUCTION AND NAVIGATION

(71) Applicant: Peter T. Zavislak, Carmel, IN (US)

(72) Inventor: Peter T. Zavislak, Carmel, IN (US)

(21) Appl. No.: 19/655,490

(22) Filed: Apr. 22, 2026

Related U.S. Application Data
(60) Provisional application No. 63/792,859, filed on Apr. 22, 2025.

(51) Int. Cl. A61B 90/00; G06T 13/20; G06T 15/00; G06V 10/98; G06V 20/40; G06V 20/50

(52) U.S. Cl. CPC A61B 90/37; G06T 13/20; G06T 15/00; G06V 20/41; G06V 20/50; A61B 2090/367; A61B 2090/376; G06T 2200/24; G06T 2210/22; G06T 2210/41; G06V 10/993; G06V 2201/034

(57) ABSTRACT

Systems and methods for enhanced intraoperative visualization are provided including an interface, a processor, a display, and a control mechanism. The interface is adapted to receive a sequence of medical images of an operational area from an imaging device. The processor is adapted to generate, from at least a portion of the medical images, a three-dimensional representation of the operational area. The display is adapted to present a first view layer comprising the sequence of medical images of the operational area and a second view layer comprising the three-dimensional representation of the operational area. The second view layer is overlaid on and spatially aligned with the first view layer. The control mechanism is adapted to fade an opacity of the second view layer.

### CROSS-REFERENCE TO RELATED APPLICATIONS

This application claims priority of Provisional Application Ser. No. 63/792,859 filed Apr. 22, 2025, entitled “System and Method for Digital Anatomical Reconstruction and Navigation.”

### TECHNICAL FIELD

This disclosure relates generally to medical imaging and visualization systems. More particularly, this disclosure relates to medical imaging and visualization systems which employ three-dimensional representations of anatomy for intraoperative use.

### BACKGROUND

The statements in this section merely provide background information related to the present disclosure and may not constitute prior art.

Live video medical imaging is commonly used during medical procedures to visualize the operational area such that the operator can accurately and safely provide necessary treatment to a patient. The most common method of providing live video medical imaging is through the use of a mobile fluoroscopy machine, often called a “C-Arm,” which allows X-ray fluoroscopic visualization of an operational area at multiple angles and positions. However, for nuanced anatomy, such as the spine, X-ray images can often be unclear and therefore difficult to navigate using medical devices.

Methods exist of building three-dimensional representations of the operational area which may be more clear for navigation. However, the accuracy of current three-dimensional representations are difficult for an operator to verify in the midst of the procedure. Furthermore, as a procedure advances, the anatomy may change as fluid is injected, tissue is moved or removed, medical devices are implanted, or many other possible changes occur. Each of these changes may render a pre-established three-dimensional representation inaccurate for further usage. Additionally, three-dimensional representations of operational areas frequently cannot provide accurate positional overlays of the medical devices being used by the operator within the operational area.

For at least these reasons, it is desirable that a method of visualization including a three-dimensional representation provide some method for the operator to verify the accuracy of the three-dimensional representation. Furthermore, it is desirable that the method of visualization including a three dimensional representation is capable of automatically detecting a change in the live operational area and updating the three dimensional representation accordingly. Furthermore, it is desirable that a three-dimensional representation of an operational area provides an accurate positional overlay of relevant medical devices within the operational area.

### SUMMARY

Further areas of applicability will become apparent from the description provided herein. It should be understood that the description and specific examples are intended for purposes of illustration only and are not intended to limit the scope of the present disclosure.

In one embodiment, a system for enhanced intraoperative visualization is provided including an interface, a processor, a display, and a control mechanism. The interface is adapted to receive a sequence of medical images of an operational area from an imaging device. The processor is adapted to generate, from at least a portion of the medical images, a three-dimensional representation of the operational area. The display is adapted to present a first view layer comprising the sequence of medical images of the operational area and a second view layer comprising the three-dimensional representation of the operational area. The second view layer is overlaid on and spatially aligned with the first view layer. The control mechanism is adapted to fade an opacity of the second view layer.

In another embodiment, a system for enhanced intraoperative visualization is provided including a fluoroscopic imaging device, an interface, a processor, a display, and a control mechanism. The fluoroscopic imaging device is adapted to create a fluoroscopic video feed of an operational area comprising a sequence of medical images. The interface is adapted to receive the sequence of medical images from the fluoroscopic imaging device. The processor is adapted to produce a sequence of processed images by modifying each of the sequence of medical images. The processor is also adapted to generate, from at least a portion of the processed images, a three-dimensional representation of the operational area. The display is adapted to present a first view layer comprising the sequence of medical images of the operational area and a second view layer comprising the three-dimensional representation of the operational area. The second view layer is overlaid on and spatially aligned with the first view layer. The control mechanism is adapted to fade an opacity of the second view layer.

In yet another embodiment, a method for enhanced intraoperative visualization is provided including receiving a sequence of medical images of an operational area from an imaging device through an interface, generating a three-dimensional representation of the operational area, presenting on a display including a first view layer and a second view layer, and fading an opacity of the second view layer using a control mechanism. Generating the three-dimensional representation of the operational area is performed by the processor from at least a portion of the medical images. The first view layer includes the sequence of medical images of the operational area. The second view layer includes the three-dimensional representation of the operational area. The second view layer is overlaid on and spatially aligned with the first view layer.

### BRIEF DESCRIPTION OF THE DRAWINGS

The embodiments may be better understood with reference to the following drawings and description. The components in the figures are not necessarily to scale. Moreover, in the figures, like-referenced numerals designate corresponding parts throughout the different views.

FIG. 1 illustrates an example of a perspective view of a medical visualization system system including an imaging device, an interface, and a display;

FIG. 2 illustrates an example of a medical image including an operational area, metadata, and a cropped area;

FIG. 3 illustrates an example of the medical image of FIG. 2 including the operational area;

FIG. 4 illustrates a front plan view of a first example of a display including a first view layer, a second view layer, an operational area, a three-dimensional representation, and a control mechanism;

FIG. 5 illustrates a front plan view of a second example of a display including a first view layer, a second view layer, an operational area, a three-dimensional representation, and a control mechanism;

FIG. 6 illustrates a front plan view of a third example of a display including a first view layer, a second view layer, a third view layer, a medical device, an operational area, a three-dimensional representation, and a control mechanism;

FIG. 7 illustrates a front plan view of a fourth example of a display including a first view layer, a second view layer, an operational area, a three-dimensional representation, and a control mechanism;

FIG. 8 illustrates a graph for an automated fading and restoring cycle for an overlaid three-dimensional representation as a function of time.

FIG. 9 illustrates a first example of a flow diagram of operations for enhanced intraoperative visualization;

FIG. 10 illustrates a second flow diagram of operations for enhanced intraoperative visualization; and

The drawings described herein are for illustration purposes only and are not intended to limit the scope of the present disclosure in any way.

### DETAILED DESCRIPTION

The following description is merely exemplary in nature and is not intended to limit the present disclosure, application, or uses.

In one example, a system for enhanced intraoperative visualization is provided including an interface, a processor, a display, and a control mechanism. The interface is adapted to receive a sequence of medical images of an operational area from an imaging device. The processor is adapted to generate, from at least a portion of the medical images, a three-dimensional representation of the operational area. The display is adapted to present a first view layer comprising the sequence of medical images of the operational area and a second view layer comprising the three-dimensional representation of the operational area. The second view layer is overlaid on and spatially aligned with the first view layer. The control mechanism is adapted to fade an opacity of the second view layer.

One technical advantage of the systems and methods described below may be that the visualization system may be used by the operator to easily verify the accuracy of the three-dimensional representation against live fluoroscopy. Another technical advantage of the systems and methods described below may be that the visualization system may be capable of automatically detecting a change in the live operational area and then update the three dimensional representation accordingly. Yet another technical advantage of the systems and methods described below may be that the visualization may provide an accurate positional overlay of relevant medical devices within the operational area.

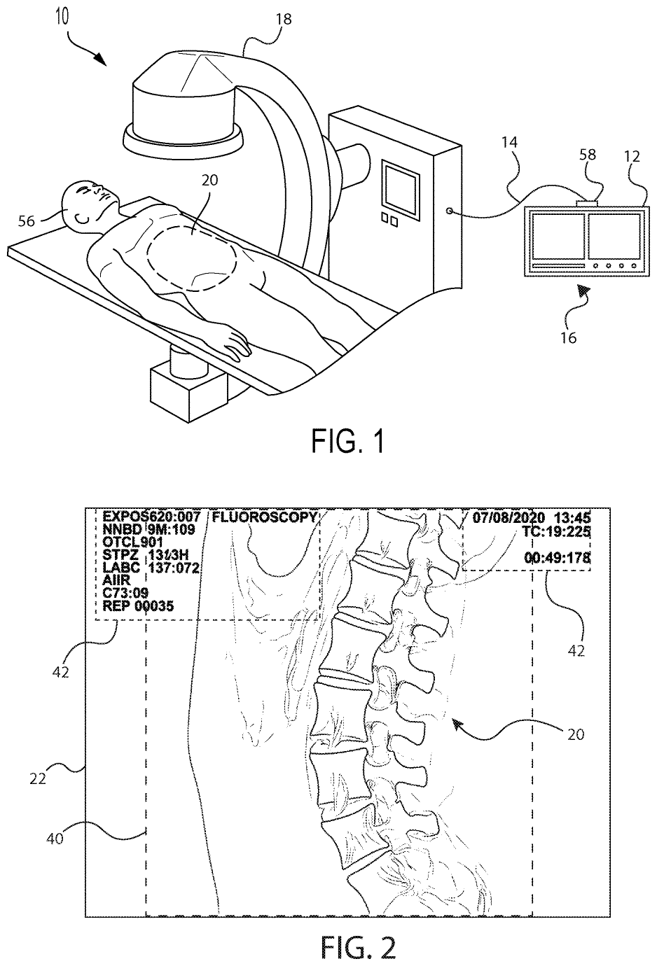

FIG. 1 illustrates a perspective view of a medical visualization system 10 deployed in a clinical environment to acquire image data of an operational area 20 of a patient 56 by an imaging device 18 and to present, via an interface 14, visualization outputs used during a procedure onto a display 16. The imaging device 18 may be positioned in view of the operational area 20 of the patient 56 such that medical images (22 in FIG. 2) may be captured of an internal anatomical structure of the operational area 20 at a variety of angles and orientations. Examples of the imaging device 18 may include a C-Arm, a fixed fluoroscopy system, or a cone-beam computed tomography device. The operational area 20 may be any portion of the patient 56 upon which a medical procedure is to be performed. Examples of the operational area 20 may include the head, spine, lungs, heart of the patient 56.

The display 16 may be coupled to the imaging device 18 through the interface 14. The display 16 may be any portion of the medical visualization system 10 which is capable of presenting various views of the operational area 20. Examples of the display 16 may include a computer with an attached monitor, a projector capable of projecting on a wall, or a tablet computer. In some embodiments the display 16 may be integral to the imaging device 18.

The interface 14 may be any portion of the medical visualization system 10 which may be adapted to transmit medical images (22 in FIG. 2) from the imaging device 18 to any other relevant components, such as a processor 12 or a display 16. Examples of the interface 14 may include a video cable, a wireless data connection, or any other electrical transmission method. In some embodiments, such as when the display 16 is integral to the imaging device 18, the interface 14 may be internal within the imaging device 18. The interface 14 may include an interface port 58, which may be any portion of the interface 14 which couples a video or data cable from the imaging device 18 to the processor 12 or display 16. For example, the interface port 58 may be capable of simultaneously receiving a variety of cable connections such as High-Definition Multimedia Interface (HDMI™), video graphics array (VGA), display port (DP), digital visual interface (DVI), or serial digital interface (SDI). The interface port 58 may include connections for one or many different video or data standards.

The processor 12 may be any portion of the medical visualization system 10 which is electronically coupled to the imaging device 18 through the interface 14 and to the display 16. The processor 12 may be a computing device which is integral to the display 16 or may be a remote server in connection with the interface 14 and the display 16 through an internet connection. Alternatively, the processor 12 may include portions which are integral to the display 16 and portions which are a remote server. An internet-based processor 12 may be advantageous to remote facilities who may not be able to afford the purchase and upkeep of complicated computing equipment.

The imaging device 18 may be adapted to create a sequence of medical images 22 of the operational area 20 at a variety of angles and orientations. The sequence of medical images 22 may be a collection of individual sequenced fluoroscopic pictures or may be a continuous fluoroscopic video feed of the operational area 20. The imaging device 18 may produce a continuously updating sequence of medical images 22 that may be used throughout the medical procedure. The interface 14 may be electronically coupled to the imaging device 18 to receive the sequence of medical images 22 and transmit them to the display 16, the processor 12, or both.

FIG. 2 illustrates an example of one of the sequence of a medical images 22 of the operational area 20. The processor 12 may be adapted to generate, from at least a portion of the medical images 22, a three-dimensional representation 24 of the operational area 20. However, in many embodiments, the medical image 22 from the imaging device 18 may include extraneous information. Extraneous information may be any portion of the medical image 22 which is not needed or which is adverse to the generation of a three-dimensional representation 24. Examples of extraneous information may include noise (44 in FIG. 6) or metadata 42, which as illustrated in FIG. 2, refers to visual elements that the fluoroscopy machine might display to the clinician, such as burned-in patient information, exposure settings, or timestamps, that would be considered noise to the machine learning model. Furthermore, the medical image 22 may include portions which are not relevant to the operational area 20. These portions may be removed as lying outside of a cropped region 40 of the operational area 20.

As illustrated in FIG. 3, the processor 12 may be adapted to produce a sequence of processed images 38 from each of the sequence of medical images 22. In this production, the processor 12 may automatically detect and isolate the operational area 20 within the medical image 22 by removing portions of the medical image 22 outside of the cropped region 40 from the processed image 38. Furthermore, the processor 12 may automatically detect and remove extraneous information, such as the metadata 42 in FIG. 3. During this production, the processor 12 may automatically discard a medical image 22 from the sequence of medical images 22 if the medical image fails to satisfy a quality criterion. Such quality criterion may include motion blur, underexposure, or occlusion.

Frequently, medical images 22 from the same imaging device 18 may be quite similar with respect to metadata 42 or noise 44. Therefore, in some embodiments, once the extraneous information has been detected and the operational area 20 isolated by the processor 12 for a single medical image 22, the processor 12 may create a template to use in producing the whole sequence of processed images 38. The template may be useful to the processor 12 in hastening the production of the sequence of processed images 38 and may reduce computational overhead on the processor 12.

The processor 12 may then generate the three-dimensional representation 24 of the operational area 20 from at least a portion of the sequence of processed images 38. In some embodiments, the processor 12 may incorporate artificial intelligence computing methods to construct the three-dimensional representation 24. For example, image translation, three-dimensional morphable models (3DMM), deep learning-based depth estimation, generative adversarial networks, self-supervised neural networks, vision transformers, contrastive unpaired translation, Stable Diffusion®-based models, and synthetic computed tomography methods (CT) methods may all be used to quickly construct the three-dimensional representations 24 from the two-dimensional medical images 22 captured by the imaging device 18. Once constructed, the processor 12 may transmit the three-dimensional representation 24 of the operational area 20 to the display 16. Because the three-dimensional representation 24 must be generated under live operational circumstances, it may be critical that the three-dimensional representation 24 is generated quickly, taking no more than 5 seconds and ideally less than 1 second. Due to this constraint, faster models including but not limited to Pix2PixHD may be more suitable than other models.

Furthermore, in some embodiments, the processor 12 may employ single-image 3D view synthesis (monocular 3D reconstruction) to generate the three-dimensional representation 24 from a single static medical image 22. This may be achieved using machine-learning models trained on large paired datasets-such as corpuses of two-dimensional fluoroscopic images paired with corresponding ground-truth three-dimensional volumetric scans (e.g., CT or magnetic resonance imaging (MRI) surfaces). Through this paired training, the neural network may learn deep anatomical priors, allowing the neural network to infer and reconstruct complex three-dimensional geometry, hidden volumetric depth, and mesh surfaces from a single two-dimensional projection without requiring a continuous fluoroscopic sweep.

To accommodate continuous advancements in artificial intelligence, the visualization system 10 may employ a modular, model-agnostic software architecture. The processor 12 may execute the artificial intelligence computing methods as an interchangeable inference engine, microservice, or containerized module. This permits the system to dynamically update, hot-swap, or replace the active image-to-mesh machine-learning model with newly discovered or superior algorithmic modalities via over-the-air (OTA) updates or application program interfaces (API), without altering the underlying user interface or spatial alignment mechanisms. The processor 12 may be adapted to map input two-dimensional radiographic data to the output three-dimensional representation 24, ensuring compatibility with future generative models.

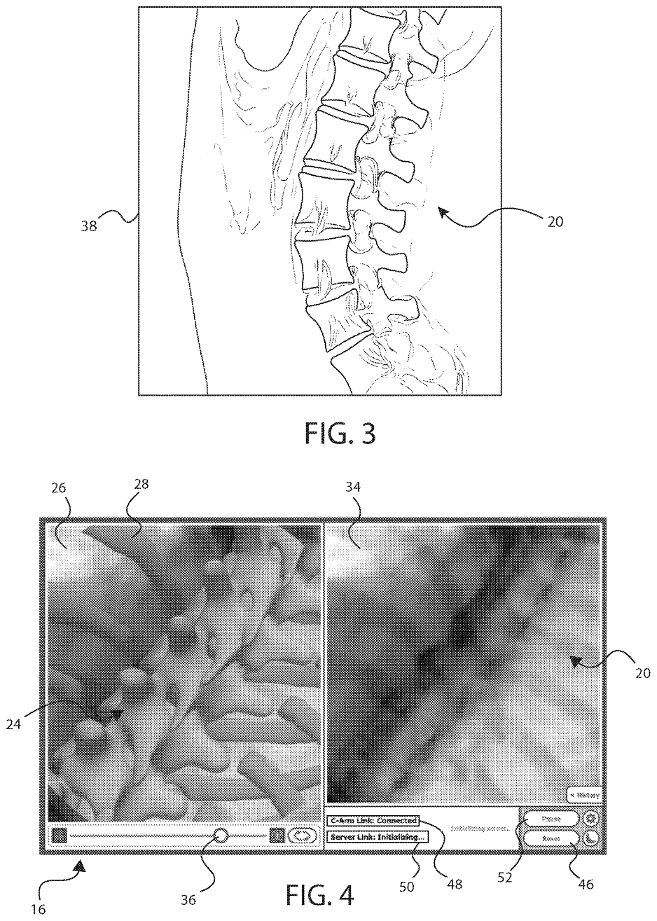

In further embodiments, to enhance spatial awareness and depth perception, the processor 12 may generate a synthetic spatiotemporal animation or pseudo-three-dimensional (4D) visualization from a plurality of intermittent medical images 22. For example, when an operator captures multiple temporally or spatially distinct medical images 22 (such as sequential steps of instrument insertion or slightly different projection angles), the processor 12 may aggregate a plurality of intermittently captured medical images 22 from the sequence of medical images 22. The processor 12 may then utilize image interpolation techniques such as algorithmic interpolation, optical flow, or generative video-synthesis machine-learning models (e.g., temporal view synthesis or deep frame interpolation) to generate synthetic intermediate frames between the captured medical images 22. This may computationally stitch the discrete images together, allowing the display 16 to generate a continuous, interpolated kinematic animation demonstrating the simulated rotation or movement of the anatomical mesh-surface in conjunction with the kinematic trajectory of the medical instruments 32. This dynamic motion may provide the clinician with motion parallax, drastically improving the perception of complex depth relationships. Furthermore, to ensure clinical safety, the visualization system 10 may include a validation interface adapted to allow an operator or assistant to independently validate the ground-truth captured keyframe medical images 22 prior to the processor 12 generating the fully interpolated animation. The control mechanism (36 in FIG. 4) may further include a timeline, scrubbing interface, or rotational control to manually manipulate, reverse, or scrub through continuous, interpolated kinematic animation.

FIG. 4 illustrates an example of the display 16. In some embodiments, the display 16 may present multiple visualization panes for intraoperative viewing of the operational area 20. For example, as illustrated in FIG. 4, the display 16 may include a left pane presenting a first view layer 26 and a second view layer 28 and an adjacent right pane presenting a video view layer 34. The first view layer 26 may be any view layer which includes the sequence of medical images 22 or the sequence of processed images 38 of the operational area 20. The sequence of medical images 22 may be included in the first view layer 26 without any production of processed images 38 from the processor 12. Alternatively, the first view layer 26 may only include the sequence of processed images 38.

Additionally, the second view layer 28 may include the three-dimensional representation 24 of the operational area 20. The three-dimensional representation 24 may be overlaid on the first view layer 26 and spatially aligned with the first view layer 26. As a result, the second view layer 28 may be visually coherent with the first view layer 26 to the operator. Furthermore, the second view layer 28 may have an opacity (60 in FIG. 8). As the second view layer 28 may be spatially aligned with the first view layer 26, the opacity 60 of the second view layer may be relative to the first view layer 26. Because the processor 12 may utilize direct image-to-image translation models, the newly generated second view layer 28 may be applied directly over the underlying source structure of the first view layer 26. As a result, the spatial alignment between the second view layer 28 and the first view layer 26 may be inherently preserved with nearly 1:1 pixel accuracy, eliminating the need for complex secondary spatial registration.

The fading and restoring of the opacity 60 of the second view layer 28 may be any effect which adjusts the visibility of the second view layer 28 over the first view layer 26. For example, in some embodiments, the control mechanism 36 may fade or restore the opacity 60 of the second view layer 28 by controlling a heat-map transition, a line-art layer wipe, a localized “magic window” window effect, or any other visual blending effect.

The video view layer 34 may be any view layer which depicts the most currently imaged view of the operational area 20 taken by the imaging device 18. In some embodiments, the video view layer 34 may be a live video feed of the fluoroscopy taken by the imaging device 18. Furthermore, in some embodiments the video view layer 34 may be identical to the first view layer 26. Alternatively, the in some embodiments, the video view layer 34 may include the unprocessed sequence of medical images 22 from the imaging device 18 while the first view layer 26 may include the sequence of processed images 38.

The display 16 may further include a control mechanism 36 adapted to fade the opacity 60 of the second view layer 28 relative to the first view layer 26. The control mechanism 36 may be any part of the display 16 which sets and controls the opacity 60 of the second view layer 28 while overlaid with the first view layer 26. Examples of the control mechanism 36 may include a sliding control, a dial, a touchscreen mechanism, or may be voice activated. A sterile cover may be placed over the control mechanism 36 to allow the operator to maintain strict sanitary practices while manipulating the control mechanism 36. Manipulation of the control mechanism 36 may cause an alpha blending parameter associated with the second view layer 28 to be modified such that the three-dimensional representation 24 transitions between increased visibility and reduced visibility while the first view layer 26 remains viewable. Furthermore, the control mechanism 36 may provide a continuous range of opacity states and can be used during a procedure to manage the operator's view of the second view layer 28 in relation to the first view layer 26. The control mechanism 36 may response to a user selection to selectively fade the second view layer 28.

The first view layer 26 and the overlayed second view layer 28 being adjacent to the video view layer 34 may be useful to the operator during a procedure. For example, while the opacity 60 of the second view layer 28 is high, between 70% to 100% and ideally around 80%, the operator can clearly discern complex anatomical features in the three-dimensional representation 24 that may not be as clear in the video view layer 34. Additionally, the operator may be easily able to compare the accuracy of the three-dimensional representation 24 within the operational area by observing and comparing it to the adjacent video view layer 34. The positions of the adjacent panes within the display 16 may be in any arrangement. For example in another embodiment, the first view layer 26 and the second view layer 28 may be presented in a right pane while the video view layer 34 may be presented in a left pane.

The display 16 may include additional information regarding the status of the medical visualization system 10. For example, the display 16 may include an imaging connection portion 48 to verify the electronic link and data exchange between the imaging device 18 and the display 16 through the interface 14. The display 16 may also include a server status portion 50 which may indicate if the processor 12 is in communication with an off-site portion.

The display 16 may also include additional control interfaces. For example, in some embodiments, the display 16 may include a pause button 52 which may allow the operator to either pause the medical visualization system 10 or pause the video view layer 34. Furthermore, the display 16 may include a reset button 46 which may allow the operator to either refresh the display 16 or may be used to force the processor 12 to update the three-dimensional representation 24 from an updated sequence of medical images 22.

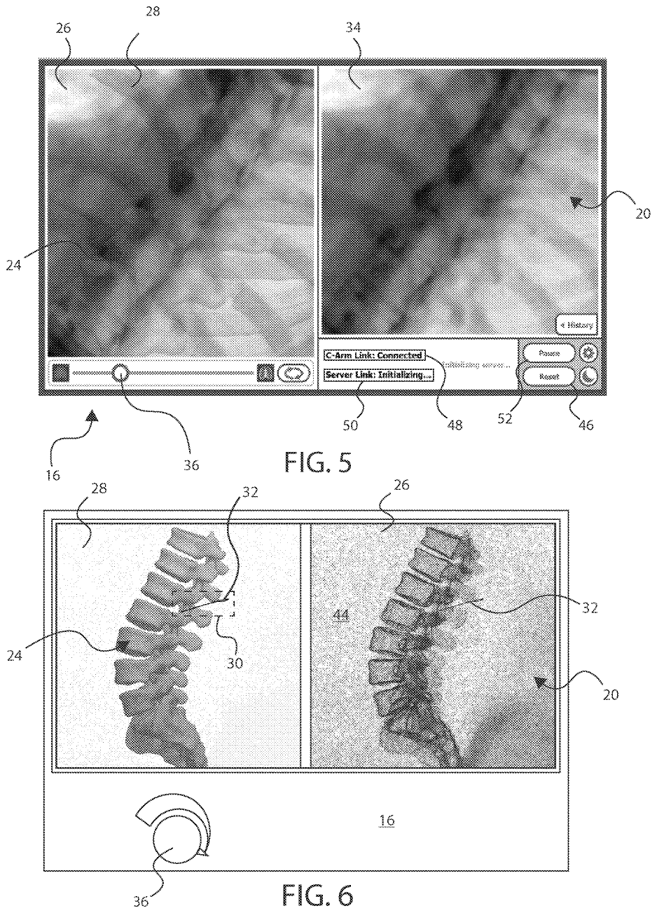

FIG. 4 illustrates the display 16 with the second view layer 28 in a high opacity presentation with the control mechanism 36 in a first position. FIG. 5 illustrates the display 16 with the second view layer 28 in a low opacity presentation with the control mechanism 36 in a second position. While the opacity 60 of the second view layer 28 is low, between 0% and 30% and ideally about 20%, the operator may be able to compare the edges of the three-dimensional representation 24 with the underlying medical images 22 to verify accuracy, or may be able to verify the accuracy of the processed images 38 to the medical images 22 displayed in the video view layer 34.

FIG. 6 illustrates another example of the display 16 including a medical instrument 32 visible in the operational area 20. The medical instrument 32 may be any item used by the operator within the operational area 20 during the procedure. Examples of the medical instrument 32 may include a needle, a catheter, contrast fluid, or a fiducial. The medical instrument 32 may be easily visible in the sequence of medical images 22 due to high density relative to the environment. However, in some embodiments, the processor 12 may diminish or eliminate the medical instrument 32 in the sequence of processed images 38. Therefore, the processor 12 may also detect the medical instrument 32. To further aid in visualization, the processor 12 may automatically recognize the medical instrument 32 by detecting specific fluoroscopic-detectable markings. Additionally, the processor 12 may automatically recognize and track the medical instrument 32 when specifically a fiducial marker within the operational area 20 based on specific pre-defined geometric profiles or shapes.

The processor 12 may also generate an enhanced view of the medical instrument 32 within a third view layer 30. The display 16 may incorporate the third view layer 30 overlaid with and spatially aligned with the first view layer 26 and the second view layer 28 such that the operator may see the position of the medical instrument 32 within the operational area 20 even when the opacity 60 of the second view layer 28 is high. In some embodiments the third view layer 30 may have an opacity of no greater than 80% to prevent the medical instrument 32 from completely obscuring critical portions of the operational area 20.

In addition to standard level, brightness, and contrast image manipulations, the processor 12 may utilize advanced generative technologies to augment the appearance of bones and instruments within the sequence of processed images 38 or within the first view layer 26, second view layer, and third view layer. For example, spatial conditioning models such as a Lineart ControlNet may be used to generate an edge-enhanced or line-art layer that explicitly highlights anatomical contours and boundaries for items in the operational area such as the medical instrument 32. This generated line-art layer may be incorporated into the second view layer 28 or the third view layer 30 to provide superior structural clarity.

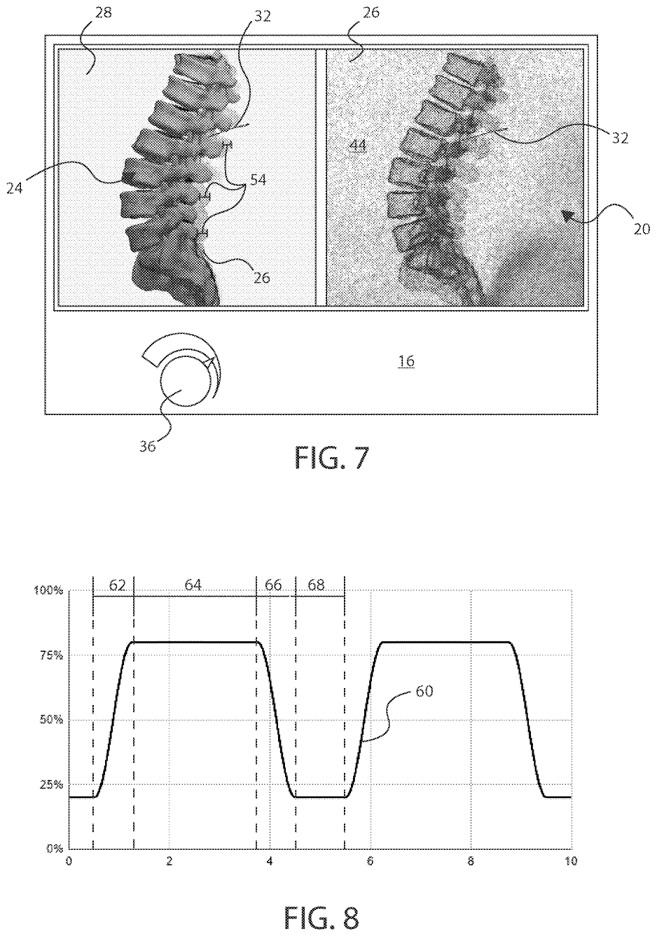

FIG. 7 illustrates another example of the display 16. In the course of a procedure, the operational area 20 may change due to a variety of factors. For example, anatomy may shift, the medical instrument 36 may be moved, tissue may be cut or altered, or fluid may be injected. An accurate and updated three-dimensional representation 24 may be critical to the success of the medical procedure. Therefore, in some embodiments, the processor 12 may periodically compare one or more of the sequence of medical images 22 used to generate the three-dimensional representation 24 to one or more updated images of an updated sequence of medical images 22. If a significant visual variance 54 is detected, the processor 12 may update the three-dimensional representation 24 to ensure continue fidelity between the first view layer 26 and the second view layer 28. The processor 12 may compare older medical images 22 to updated medical images between 1 to 60 times per second, and ideally about 30 times per second.

However, updating the three-dimensional representation 24 too frequently may cause a shaking or jittering effect on the second view layer 28 and thereby render the display 16 less useful. For example, due to random variations such as noise, every medical image 22 may be visually distinct from any other medical image 22 but small differences between each medical image 22 may not require an update of the three-dimensional representation 24. For this reason, the processor 12 may compute a visual difference metric between at least one medical image 22 in the sequence and at least one medical image 22 in an updated sequence of medical images 22. If the computed visual difference metric exceeds a pre-determined threshold value, the processor 12 may generate an updated three-dimensional representation 24 of the operational area 20 using at least a portion of the updated sequence of medical images 22.

In some embodiments, the processor 12 may be adapted to generate an updated three-dimensional representation 24 of the operational area 20 based on other criteria. For example, in some embodiments, the processor 12 may update the three-dimensional representation 24 based on a pre-defined timing criteria of no more than 1 minute, but ideally between 5-10 seconds, from when the last three-dimensional representation 24 was generated. Alternatively, the operator may force the processor 12 to generate an updated three-dimensional representation 24. In some embodiments, the imaging device 18 may transmit a signal to the processor 12 indicating a change in orientation of the imaging device 18, such as tilting, rotating, or translating. When such a signal is received by the processor 12, or when a change in orientation of the imaging device 18 is detected by the processor 12, the processor 12 may generate an updated three-dimensional representation 24 of the operational area 20 from an updated sequence of medical images 22.

FIG. 8 illustrates a graph showing an automatic and cyclical fading and restoring of the opacity 60 of the second view layer 28 overlaid on the first view layer 26. In some embodiments, the control mechanism 36 automatically fade and restore the opacity 60 of the second view layer 28 according to a timed cycle. This may created a pulsing effect which may be useful to the operator in verifying the accuracy of the three-dimensional representation 24 without having to selectively control the opacity 60. In many medical procedures, an operator must maintain strict sanitary conditions. In such circumstances, the automated timed cycle may prevent the operator from having to touch the control mechanism 36 and thereby risk contamination.

The cycle of the opacity 60 over time may include four periods: a fade in period 62, a high opacity period 64, a fade out period 66, and a low opacity period 68. After the low opacity period 68, the cycle would begin again. The fade in period 62 may be a smooth curvature transition, changing from a low opacity 60 to a high opacity in no more than 1 second and ideally about 0.75 seconds. The high opacity period 64 should be the longest period in the cycle, lasting between 2.0 to 3.0 seconds, and ideally about 2.5 seconds. Over many cycles, most of the procedure may take place in the high opacity period 64. The fade out period 66 may be a smooth curvature transition, changing from a high opacity 60 to a low opacity in no more than 1 second and ideally about 0.75 seconds. The low opacity period 68 should be long enough for the operator to gauge the accuracy of the three-dimensional representation 24 relative to the medical image 22, between 1.0 to 2.0 seconds, ideally about 1.0 seconds. The whole cycle should take between 4 to 6 seconds, ideally about 5 seconds.

Furthermore, although specific components are described above, methods, systems, and articles of manufacture described herein may include additional, fewer, or different components. For example, multiple separate processors 12 may be used for each function performed by the processor 12. Furthermore, multiple displays 16 may be used.

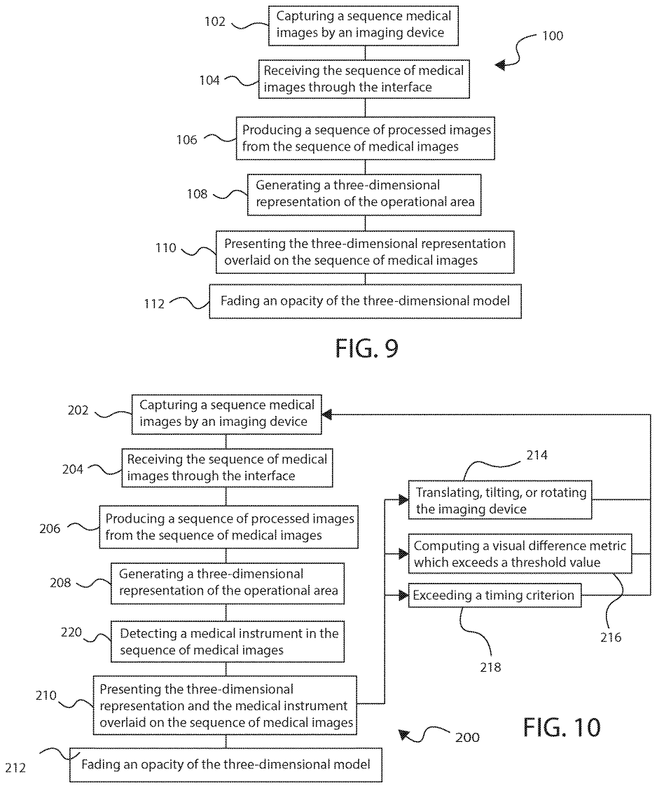

FIG. 9 illustrates a flow diagram of operations for enhanced intraoperative visualization. The operations may include fewer, additional, or different operations than illustrated in FIG. 9. Alternatively or in addition, the operations may be performed in a different order than illustrated.

The visualization process (100) may be performed by initially capturing the sequence of medical images 22 of the operational area 20 by the imaging device 18 (102). The processor 12 or the display 16 may receive the sequence of medical images through the interface 14 (104). The processor 12 may produce the sequence of processed images 38 from the sequence of medical images 22 (106). The processor 12 may also generate the three-dimensional representation 24 of the operational area 20 from a portion of the sequence of medical images 22 or from the sequence of processed images 38 (108). The display 16 may present the second view layer 28 including the three-dimensional representation 24 overlaid on the first view layer 26 including the sequence of medical images 22 or the sequence of processed images 38 (110). The control mechanism 36 may be used to fade the opacity 60 of the second view layer 28 including the three-dimensional representation 24 (112).

FIG. 10 illustrates another flow diagram of operation for enhanced intraoperative visualization. In some embodiments, the visualization process (200) may include the steps discussed in FIG. 9 regarding capturing the sequence of medical images 22 by the imaging device 18 (202), receiving the sequence of medical images 22 through the interface 14 (204), producing the sequence of processed images 38 from the sequence of medical images 22 (206), and generating the three-dimensional representation 24 of the operational area 20 (208). The visualization process (200) may also include detecting the medical instrument 32 in the sequence of medical images 22 by the processor 12 (220). The display 16 may present the second view layer 28 including the three-dimensional representation 24 and the third view layer 30 including the medical instrument 32 overlaid on the first view layer 26 including the sequence of medical images 22 or the sequence of processed images 38 (210). The control mechanism 36 may be used to fade the opacity 60 of the second view layer 28 including the three-dimensional representation 24 (212).

Furthermore, the visualization process (200) may include several ongoing processes during the procedure. In each of these processes, the processor 12 may repeat the process of generating an updated three-dimensional representation 24 of the operational area 20 from an updated sequence of medical images 22 (208). For example, the visualization process (200) may include translating, tilting, or rotating the imaging device 18 (214), the processor 12 comparing a medical image 22 to an updated medical image 22 and computing a visual difference metric which exceeds a threshold value (216), or exceeding a pre-defined timing criterion since the three-dimensional representation 24 was last generated (218). Each of these processes (214, 216, 218) may cause the visualization process (200) to loop back to an earlier step.

In addition to the advantages that have been described, it is also possible that there are still other advantages that are not currently recognized but which may become apparent at a later time. While various embodiments have been described, it will be apparent to those of ordinary skill in the art that many more embodiments and implementations are possible. Accordingly, the embodiments described herein are examples, not the only possible embodiments and implementations.

I claim:

1. A system for enhanced intraoperative visualization, comprising:
an interface adapted to receive a sequence of medical images of an operational area from an imaging device;
a processor adapted to generate, from at least a portion of the sequence of medical images, a three-dimensional representation of the operational area;
a display adapted to present a first view layer comprising the sequence of medical images of the operational area and a second view layer comprising the three-dimensional representation of the operational area, wherein the second view layer is overlaid on and spatially aligned with the first view layer; and
a control mechanism adapted to fade an opacity of the second view layer.

2. The system of claim 1, wherein the sequence of medical images is a fluoroscopic video feed.

3. The system of claim 2, wherein:
the processor is adapted to produce a sequence of processed images by detecting the operational area, isolating the operational area, and removing extraneous information from each of the sequence of medical images; and
the processor is adapted to generate, from at least a portion of the processed images, the three-dimensional representation of the operational area.

4. The system of claim 1, wherein the processor is adapted to update the three-dimensional representation of the operational area based on an updated sequence of medical images in accordance with a timing criterion.

5. The system of claim 4, wherein:
the processor is adapted to compute a visual difference metric between at least one medical image in the sequence and at least one medical image in the updated sequence; and
the processor is adapted to update the three-dimensional representation of the operational area if the visual difference metric exceeds a threshold value.

6. The system of claim 1, wherein the display further comprises a video view layer comprising a continuously updating sequence of medical images received from the imaging device and the video view layer is positioned adjacent to overlaid first view layer and second view layer.

7. The system of claim 1, wherein the control mechanism is adapted to selectively fade the opacity of the second view layer overlaid on the first view layer in response to user selection.

8. The system of claim 1, wherein the control mechanism is adapted to automatically fade and restore the opacity of the second view layer overlaid on the first view layer according to a timed cycle.

9. A system for enhanced intraoperative visualization, comprising:
a fluoroscopic imaging device adapted to create a fluoroscopic video feed of an operational area comprising a sequence of medical images;
an interface adapted to receive the sequence of medical images from the fluoroscopic imaging device;
a processor adapted to produce a sequence of processed images by modifying each of the sequence of medical images and to generate, from at least a portion of the processed images, a three-dimensional representation of the operational area;
a display adapted to present a first view layer comprising the sequence of medical images of the operational area and a second view layer comprising the three-dimensional representation of the operational area, wherein the second view layer is overlaid on and spatially aligned with the first view layer; and
a control mechanism adapted to fade an opacity of the second view layer.

10. The system of claim 9, wherein the processor is adapted to detect a medical instrument in the sequence of medical images of the operational area, and the display further comprises a third view layer comprising an enhanced view of the medical instrument detected by the processor and the third view layer is overlaid on and spatially aligned with the first view layer and the second view layer.

11. The system of claim 10, wherein the processor is adapted to discard a medical image from the sequence of medical images if the medical image fails to satisfy a quality criterion including at least one of motion blur, underexposure, or occlusion.

12. The system of claim 10, wherein:
the processor is adapted to aggregate a plurality of intermittently captured medical images from the sequence of medical images, produce synthetic intermediate frames between the plurality of intermittently captured medical images using an image interpolation technique, and generate a continuous kinematic animation; and
the display is adapted to present the movement of the medical instrument relative to the three-dimensional representation within the operational area.

13. The system of claim 12, further comprising a validation interface adapted to allow an operator to validate the captured medical images prior to generation of the continuous kinematic animation.

14. A method for enhanced intraoperative visualization utilizing an imaging device, a processor, an interface, a display, and a control mechanism, the method comprising:
receiving a sequence of medical images of an operational area from the imaging device through the interface;
generating, by the processor and from at least a portion of the medical images, a three-dimensional representation of the operational area;
presenting on the display comprising a first view layer and a second view layer, wherein the first view layer comprises the sequence of medical images of the operational area, the second view layer comprises the three-dimensional representation of the operational area, and the second view layer is overlaid on and spatially aligned with the first view layer; and
fading an opacity of the second view layer using the control mechanism.

15. The method of claim 14, further comprising producing a sequence of processed images by detecting the operational area, isolating the operational area, and removing extraneous information from each of the sequence of medical images, wherein generating the three-dimensional representation is performed using at least a portion of the processed images.

16. The method of claim 14, further comprising detecting, by the processor, a medical instrument in the sequence of medical images of the operational area, wherein the display further comprises a third view layer comprising an enhanced view of the medical instrument and the third view layer is overlaid on and spatially aligned with the first view layer and the second view layer.

17. The method of claim 14, further comprising:
receiving an updated sequence of medical images; and
updating the three-dimensional representation of the operational area based on the updated sequence of medical images.

18. The method of claim 17, wherein updating the three-dimensional representation of the operational area takes no more than 5 seconds.

19. The method of claim 17, wherein updating occurs in accordance with a pre-defined timing criterion.

20. The method of claim 17, further comprising computing a visual difference metric, by the processor, between at least one medical image of the sequence and at least one medical image of the updated sequence, wherein updating occurs if the visual difference metric exceeds a threshold value.
